(19) United States
(12) Patent Application Publication
(10) Pub. No.: US 2004/0063684 A1
(43) Pub. Date: Apr. 1, 2004

(54) Treatment of oesophageal motility disorders and gastro-oesophageal reflux disease

(76) Inventor: Michael A. Kamm, London (GB)

Correspondence Address: BIRCH STEWART KOLASCH & BIRCH, PO BOX 747, FALLS CHURCH, VA, 22040-0747, US

(21) Appl. No.: 10/467,154

(22) PCT Filed: Jan. 24, 2002
(86) PCT No.: PCT/GB02/00310

(30) Foreign Application Priority Data
Feb. 5, 2001 (GB) 0102854.7
Feb. 5, 2001 (GB) 0102855.4
Feb. 5, 2001 (GB) 0102856.2

(51) Int. Cl. A61K 31/553; A61K 31/554

(52) U.S. Cl. 514/211.07; 514/355

(57) ABSTRACT

Smooth muscle tone modulator compositions and methods of use thereof are provided for the treatment of oesophageal motility disorders and gastro-oesophageal reflux disease. The smooth muscle tone modulators are applied topically to treat the disorder. Topical treatment reduces the risk of undesirable side effects observed in connection with oral or sublingual administration of the modulators.

The present invention relates to the use of a smooth muscle tone modulator in the manufacture of a medicament for use in the topical treatment of oesophageal motility disorders and gastro-oesophageal reflux disease (“GORD”). In particular, the invention relates to the use of a smooth muscle relaxant in the manufacture of a medicament for use in the topical treatment of oesophageal spasm, nutcracker oesophagus, non-specific oesophageal motility disorder, and other disorders of oesophageal body dysmotility and in the topical treatment of achalasia and hypertensive lower oesophageal sphincter (“LOS”) and to the use of a smooth muscle contraction stimulant (or “contractant”) in the manufacture of a medicament for use in the topical treatment of GORD.

The term “smooth muscle tone modulator” includes any pharmacologically-acceptable compound which regulates and/or adjusts smooth muscle tone and embraces smooth muscle relaxants and smooth muscle contractants. A smooth muscle relaxant includes agents that either decrease smooth muscle tone or prevent smooth muscle contraction and agents that have both of these activities. A smooth muscle contractant includes agents that either increase smooth muscle tone or prevent smooth muscle relaxation and agents that have both of these activities.

Normal oesophageal function is dependent on the integration of normal extrinsic nerve, intrinsic nerve and muscle functions. When oesophageal neuromuscular function is abnormal, a number of symptoms and clinical disorders can result. If there is a failure of normal peristalsis, food and liquid may fail to be propelled into the stomach, with the resultant sensation of blockage, pain and the regurgitation of food. This can happen without obvious cause, or can occur in association with recognised syndromes. One such recognised syndrome is diffuse oesophageal spasm which is a condition in which there is a failure of propagated peristalsis, with simultaneous contraction of oesophageal muscle along the length of the oesophagus. An extreme example of this condition is known as cork-screw oesophagus, in which there are high pressure segmenting non-propagating contractions of the oesophageal body. A further example is nutcracker oesophagus which is a condition in which peristaltic propagated contractions are preserved, but in which the oesophageal muscle contracts excessively strongly. This results in high pressure contractions which can result in pain for the sufferer.

In some patients, there is a non-specific disorder of oesophageal function that has the features of oesophageal spasm and nutcracker oesophagus in conjunction with the features of a different condition known as achalasia which is a condition in which there is a failure of propagated contractions in the body of the oesophagus associated with a high resting tone in the LOS and failure of LOS. relaxation on swallowing.

There is a separate condition in which the resting pressure in the LOS is increased, i.e. sphincter tone is increased above normal. Such a condition may be present without the other features of achalasia, so that the muscle may relax fully or partly on swallowing, and motility in the body of the oesophagus is not abnormal. This condition is known as hypertensive LOS.

The oesophageal body receives both an extrinsic (cholinergic parasympathetic) innervation and an intrinsic innervation. The traditional treatment of oesophageal motility disorders resulting from abnormal oesophageal neuromuscular function has relied on the use of oral (including sublingual) medication in the form of tablets or sprays comprising calcium channel blockers and nitric oxide (“NO”) donors, e.g. glyceryl trinitrate. However, all of these medications mediate their effect through a systemic mechanism, after absorption from the gastrointestinal (“GI”) tract into the blood stream. They are only moderately effective and suffer the problem of systemic side effects such as headaches and reduced blood pressure. Surgical myotomy has also been used although this treatment is inconvenient and painful for the sufferer.

The smooth muscle LOS receives both an extrinsic (noradrenergic and cholinergic parasympathetic) innervation and an intrinsic innervation. The extrinsic excitatory innervation consists of a sympathetic alpha-1 adrenergic innervation which is partly responsible for the maintenance of LOS tone. The extrinsic cholinergic parasympathetic innervation causes LOS relaxation. The sphincter receives further inhibitory extrinsic innervation which is beta-adrenergic, and possibly also involves alpha-2 effects. The sphincter also receives an innervation involving other neurotransmitters such as nitric oxide (“NO”), ATP, GABA and prostaglandins.

Traditional treatments of achalasia and hypertensive LOS include forceful or pneumatic dilation of the LOS with a dilating instrument. This is inconvenient for the sufferer and dilation usually has to be repeated. Oral (including sublingual) administration of NO donors and calcium channel blockers in the form of tablets or sprays has also been used. However, these treatments mediate their effect through a systemic mechanism, after absorption from the GI tract into the blood stream. They are only moderately effective, and suffer the problem of systemic side effects such as headaches and reduced blood pressure.

The traditional treatment of GORD depends on the severity of the condition. Mild cases are treatable by simply elevating the head of the sufferer when the sufferer is lying down, by dietary control and by taking antacids after meals and at bedtime. More severe cases are treated by reducing the level of gastric acid production using histamine type 2 (“H2”) blockers or proton pump inhibitors taken orally. Recently, there has been interest in developing systemically active drugs which modulate the neural control of the LOS, preventing excessive relaxations. Oral treatment is mediated by a systemic mechanism which gives rise to unwanted side effects such as headaches.

WO-A-87/04077 (Martin et al) discloses a pharmaceutical composition comprising a local anaesthetic adapted to inhibit relaxation of the LOS and a carrier therefor comprising a material adapted to float on gastrointestinal fluids contained in the stomach. The composition is designed to place the local anaesthetic in contact with the LOS or the gastric mucosa near the LOS. In the preferred embodiment, the composition comprises GAVISCON™ as the carrier. GAVISCON™ is produced by Reckitt & Coleman and described in U.S. Pat. No. 4,140,760.

According to a first aspect of the present invention, there is provided use of a smooth muscle tone modulator in the manufacture of a medicament for the topical treatment of oesophageal motility disorders and GORD.

When applied topically to the oesophagus, LOS and stomach lining, a high concentration of a smooth muscle tone modulator may be administered for local effect thereby avoiding systemic side effects. The modulator is not absorbed systemically and, if it is passed into the small bowel, it is absorbed and inactivated by the normal mechanisms of drug metabolism such as in the liver.

The medicament is suitable for application to the oesophagus, LOS and stomach lining as required and is preferably mucoadhesive. A mucoadhesive medicament is more resistant to being removed from the oesophageal wall or LOS than a non-mucoadhesive medicament. Prolonged contact of the medicament with the oesophageal body, LOS and stomach lining in this way improves the level of absorption or “uptake” of the smooth muscle tone modulator across the mucosal membrane of the oesophagus (i.e. the epithelium) or stomach or into the LOS when compared to that for a non-mucoadhesive medicament.

Preferably, the medicament is in the form of a solution, an emulsion, a gel or a foam that is swallowed by the sufferer. The medicament may comprise a polymeric matrix.

The smooth muscle tone modulator may be present in the medicament in a concentration of from 0.01 to 40 wt %.

A first preferred embodiment involves the use of a smooth muscle relaxant in the manufacture of a medicament for use in the topical treatment of oesophageal spasm, nutcracker oesophagus, non-specific oesophageal motility disorder, and other oesophageal body dysmotility syndromes.

A second preferred embodiment involves the use of a smooth muscle relaxant in the manufacture of a medicament for use in the topical treatment of achalasia and hypertensive LOS.

The smooth muscle relaxant may be a calcium channel blocker (e.g. diltiazem or nifedipine), a potassium channel opener, a nitric oxide donor (e.g. glyceryl trinitrate, isosorbide trinitrate, L-arginine, S-nitroso-N-acetylpenicillamine or nitroprusside), an adrenergic agonist (e.g. phenylephrine), a beta agonist (including a beta-2 agonist or a beta-3 agonist), a dopaminergic agonist, a prostaglandin modifier, a GABA antagonist, a glutamate antagonist, a tachykinin antagonist, capsaicin, dicyclomine or flavoxate. In addition, the relaxant may be an alpha-1 adrenergic antagonist (e.g. prazosin, phenoxybenzamide, dibenamide, doxazosin, terazosin, phentolamine, tolazoline or trimazosin), a cholinergic agent or anticholinesterase, a cholinergic agonist or a cholinomimetic agent (e.g. bethanechol). In the case of oesophageal body disorders in which there is excessive neuromuscular action, the smooth muscle relaxant may also be an anticholinergic agent (e.g. atropine or hyoscine).

A third preferred embodiment involves the use of a smooth muscle contractant in the manufacture of a medicament for use in the topical treatment of GORD.

The medicament of the third preferred embodiment may form a “raft” that floats on the surface of the stomach contents thereby not only placing the smooth muscle contractant in contact with the mucosal membrane of the stomach (preferably near the LOS) but also physically obstructing gastric reflux. For example, the medicament may comprise a bicarbonate compound that reacts with gastric acid to form carbon dioxide which helps foam the medicament.

The smooth muscle contractant may be an alpha-1 adrenergic agonist (e.g. phenylephrine), an anticholinergic agent (e.g. atropine, propantheline, emepronium, trospium, tolteridone, darifenacin, oxybutinin or hyoscine), a nitric oxide synthase (“NOS”) antagonist (e.g. L-NAME), a prostaglandin modifier, a GABA agonist (e.g. baclofen), a tricyclic antidepressant (e.g. imipramine or amitryptaline), a noradrenaline and serotonin uptake inhibitor (e.g. duloxetine), a serotonin agonist or antagonist, an opioid analogue, a dopaminergic antagonist, a beta-antagonist (including beta-2 and beta-3 antagonists), glutamate (or a related agonist) or a tachykinin antagonist.

The medicament may comprise an antacid.

According to a second aspect of the present invention, there is provided use of a composition comprising a smooth muscle tone modulator and a therapeutically acceptable mucoadhesive vehicle in the manufacture of a medicament for the topical treatment of oesophageal motility disorders and GORD. The term “therapeutically acceptable mucoadhesive vehicle” includes a mucoadhesive vehicle that is pharmacologically acceptable.

A first preferred embodiment of the second aspect involves the use of a composition comprising a smooth muscle relaxant and a therapeutically acceptable vehicle in the manufacture of a medicament for use in the topical treatment of oesophageal spasm, nutcracker oesophagus, non-specific oesophageal motility disorder, and other oesophageal body dysmotility syndromes.

A second preferred embodiment of the second aspect involves the use of a composition comprising a smooth muscle relaxant and a therapeutically acceptable vehicle in the manufacture of a medicament for use in the topical treatment of achalasia and hypertensive LOS.

A third preferred embodiment of the second aspect involves the use of a composition comprising smooth muscle contractant and a therapeutically acceptable vehicle in the manufacture of a medicament for use in the topical treatment of GORD.

The medicament of the preferred embodiments may be as defined above.

According to a third aspect of the present invention, there is provided a method of treating oesophageal motility disorders and GORD comprising administering topically a pharmaceutically acceptable amount of a smooth muscle tone modulator to the upper GI tract which includes the oesophagus, the LOS and the stomach.

According to fourth aspect of the present invention, there is provided a method of treating a condition selected from the group consisting of oesophageal spasm, nutcracker oesophagus, non-specific oesophageal motility disorder and other disorders of oesophageal dysmotility comprising administering topically a pharmaceutically acceptable amount of a smooth muscle relaxant to the upper GI tract, particularly the oesophagus and the LOS.

According to a fifth aspect of the present invention, there is provided a method of treating achalasia and hypertensive LOS comprising administering topically a pharmaceutically acceptable amount of a smooth muscle relaxant to the upper GI tract, particularly the oesophagus and the LOS.

According to a sixth aspect of the present invention, there is provided a method of treating GORD comprising administering topically a pharmaceutically acceptable amount of a smooth muscle contractant to the upper GI tract, particularly the LOS and the stomach.

It will be appreciated that the invention is not restricted to the details described above with reference to the preferred embodiments but that numerous modifications and variations can be made without departing from the scope of the invention as defined in the following claims.

1. Use of a smooth muscle tone modulator in the manufacture of a medicament for use in the topical treatment of oesophageal motility disorders and gastro-oesophageal reflux disease (“GORD”).

2. Use as claimed in claim 1, wherein the medicament is mucoadhesive.

3. Use as claimed in claim 1 or claim 2, wherein the medicament is in a swallowable form selected from solution, emulsion, gel or foam.

4. Use as claimed in any one of claims 1 to 3, wherein the smooth muscle tone modulator is a smooth muscle relaxant and the medicament is for use in the topical treatment of oesophageal spasm, nutcracker oesophagus, non-specific oesophageal motility disorder, and other oesophageal body dysmotility syndromes.

5. Use as claimed in any one of claims 1 to 3, wherein the smooth muscle tone modulator is a smooth muscle relaxant and the medicament is for use in the topical treatment of achalasia and hypertensive lower oesophageal sphincter (“LOS”).

6. Use as claimed in claim 4 or claim 5, wherein the smooth muscle relaxant is a calcium channel blocker.

7. Use as claimed in claim 6, wherein the calcium channel blocker is diltiazem or nifedipine.

8. Use as claimed in claim 4 or claim 5, wherein the smooth muscle relaxant is a potassium channel opener.

9. Use as claimed in claim 4 or claim 5, wherein the smooth muscle relaxant is a nitric oxide donor.

10. Use as claimed in claim 9, wherein the nitric oxide donor is glyceryl trinitrate, isosorbide trinitrate, L-arginine, S-nitroso-N-acetylpenicillamine or nitroprusside.

11. Use as claimed in claim 4 of claim 5, wherein the smooth muscle relaxant is an adrenergic agonist.

12. Use as claimed in claim 11, wherein the adrenergic agonist is phenylephrine.

13. Use as claimed in claim 4 or claim 5, wherein the smooth muscle relaxant is a beta agonist, a beta-2 agonist or a beta-3 agonist.

14. Use as claimed in claim 4 or claim 5, wherein the smooth muscle relaxant is a dopaminergic agonist.

15. Use as claimed in claim 4 or claim 5, wherein the smooth muscle relaxant is a prostaglandin modifier.

16. Use as claimed in claim 4 or claim 5, wherein the smooth muscle relaxant is a GABA antagonist.

17. Use as claimed in claim 4 or claim 5, wherein the smooth muscle relaxant is a glutamate antagonist.

18. Use as claimed in claim 4 or claim 5, wherein the smooth muscle relaxant is a tachykinin antagonist.

19. Use as claimed in claim 4 or claim 5, wherein the smooth muscle relaxant is capsaicin, dicyclomine or flavoxate.

20. Use as claimed in claim 4 or claim 5, wherein the smooth muscle relaxant is an anticholinergic agent.

21. Use as claimed in claim 4 or claim 5, wherein the smooth muscle relaxant is an alpha-1 adrenergic antagonist.

22. Use as claimed in claim 21, wherein the alpha-1 adrenergic antagonist is prazosin, phenoxybenzamide, dibenamide, doxazosin, terazosin, phentolamine, tolazoline or trimazosin.

23. Use as claimed in claim 4 or claim 5, wherein the smooth muscle relaxant is a cholinergic agent or anticholinesterase.

24. Use as claimed in claim 4 or claim 5, wherein the smooth muscle. relaxant is a cholinergic agonist.

25. Use as claimed in claim 4 or claim 5, wherein the smooth muscle relaxant is a cholinomimetic agent.

26. Use as claimed in claim 25, wherein the cholinomimetic agent is bethanechol.

27. Use as claimed in any one of claims 1 to 3, wherein the smooth muscle tone modulator is a smooth muscle contractant and the medicament is for use in the topical treatment of GORD.

28. Use as claimed in claim 27, wherein the medicament is used in a swallowable form that forms a raft that floats on the surface of the contents of the stomach.

29. Use as claimed in claim 27 or claim 28, wherein the smooth muscle contractant is an alpha-1 adrenergic agonist.

30. Use as claimed in claim 29, wherein the alpha-1 adrenergic agonist is phenylephrine.

31. Use as claimed in claim 27 or claim 28, wherein the smooth muscle contractant is an anticholinergic agent.

32. Use as claimed in claim 31, wherein the anticholinergic agent is atropine, propantheline, emepronium, trospium, tolteridone, darifenacin, oxybutinin or hyoscine.

33. Use as claimed in claim 27 or claim 28, wherein the smooth muscle contractant is a nitric oxide synthase (“NOS”) antagonist.

34. Use as claimed in claim 33, wherein the NOS antagonist is L-NAME.

35. Use as claimed in claim 27 or claim 28, wherein the smooth muscle contractant is a prostaglandin modifier.

36. Use as claimed in claim 27 or claim 28, wherein the smooth muscle contractant is a beta antagonist, a beta-2 antagonist or a beta-3 antagonist.

37. Use as claimed in claim 27 or claim 28, wherein the smooth muscle contractant is a GABA agonist.

38. Use as claimed in claim 37, wherein the GABA agonist is baclofen.

39. Use as claimed in claim 27 or claim 28, wherein the smooth muscle contractant is a tricyclic antidepressant.

40. Use as claimed in claim 39, wherein the tricyclic antidepressant is imipramine or amitryptaline.

41. Use as claimed in claim 27 or claim 28, wherein the smooth muscle contractant is a noradrenaline and serotonin uptake inhibitor.

42. Use as claimed in claim 41, wherein the uptake inhibitor is duloxetine.

43. Use as claimed in claim 27 or claim 28, wherein the smooth muscle contractant is a serotonin agonist or antagonist.

44. Use as claimed in claim 27 or claim 28, wherein the smooth muscle contractant is an opioid analogue.

45. Use as claimed in claim 27 or claim 28, wherein the smooth muscle contractant is a dopaminergic antagonist.

46. Use as claimed in claim 27 or claim 28, wherein the smooth muscle contractant is a beta-antagonist, a beta-2 antagonist or a beta-3 antagonist.

47. Use as claimed in claim 27 or claim 28, wherein the smooth muscle contractant is glutamate or a related agonist.

48. Use as claimed in claim 27 or claim 28, wherein the smooth muscle contractant is a tachykinin antagonist.

49. Use as claimed in any one of claims 27 to 48, wherein the medicament comprises an antacid.

50. Use as claimed in any one of claims 1 to 49, wherein the smooth muscle tone modulator is present in the medicament in a concentration of from 0.01 to 40 wt %.

51. Use of a composition comprising a smooth muscle tone modulator and a therapeutically acceptable mucoadhesive vehicle in the manufacture of a medicament for the topical treatment of oesophageal motility disorders and GORD.

52. Use as claimed in claim 51, wherein the smooth muscle tone modulator is a smooth muscle relaxant and the medicament is for use in the topical treatment of oesophageal spasm, nutcracker oesophagus, non-specific oesophageal motility disorder, and other oesophageal body dysmotility syndromes.

53. Use as claimed in claim 51, wherein the smooth muscle tone modulator is a smooth muscle relaxant and the medicament is for use in the topical treatment of achalasia and hypertensive LOS.

54. Use as claimed in claim 51, wherein the smooth muscle tone modulator is a smooth muscle contractant and the medicament is for use in the topical treatment of GORD.

55. A method of treating oesophageal motility disorders and GORD comprising administering topically a pharmaceutically acceptable amount of a smooth muscle tone modulator to the upper GI tract.

56. A method of treating a condition selected from the group consisting of oesophageal spasm, nutcracker oesophagus, non-specific oesophageal motility disorder and other disorders of oesophageal dysmotility comprising administering topically a pharmaceutically acceptable amount of a smooth muscle relaxant to the upper GI tract.

57. A method of treating achalasia and hypertensive LOS comprising administering topically a pharmaceutically acceptable amount of a smooth muscle relaxant to the upper GI tract.

58. A method of treating GORD comprising administering topically a pharmaceutically acceptable amount of a smooth muscle contractant to the upper GI tract.
